(19) United States
(12) Patent Application Publication
(10) Pub. No.: US 2007/0071679 A1
(43) Pub. Date: Mar. 29, 2007

(54) Methylamine risks and treatment of hyperactivity, depression, and alcoholism with epinephrine-N-quinolines

(76) Inventor: Ilkka Tapio Hilakivi, Helsinki (FI)

Correspondence Address: Ilkka Tapio Hilakivi, Runeberginkatu 4 b B 37, Helsinki, F1-00100, FI

(21) Appl. No.: 11/248,498

(22) Filed: Sep. 29, 2005

(51) Int. Cl. A61K 49/00

(52) U.S. Cl. 424/9.1

(57) ABSTRACT

Results of animal experiments proving a relationship between ontogenetically early sleep-wake behaviour and neurobehavioural disorders during later life indicate examination of ontogenetically early sleep-wake behaviour in human beings. With drugs with N-adrenaline moiety and with drugs with PNMT-inhibiting activity, in addition with effects on adrenergic beta and alpha-1 receptors, and possibly on alpha-2 receptors, during the last two intrauterine and the first four postnatal months of human ontogenesis human fetuses and babies are given treatment in order to suppress, counteract and alleviate excessive effects of pathogenetically and pathologically in the brain and body perinatally prevailing and acting methylated amines on adren-, dopamin-, noradren- and serotoninergic neurotransmissions, to treat and alleviate diseases and conditions of neurobehavioural disorders during later life such as hyperactivity in attention deficit disorder, depression, amphetamine, cocaine, ketamine, phencyclidine and opioid dependence, alcohol abuse and alcoholism and states of hyper-S-adenosyl-homocystenemia. Prophylaxis can be achieved perinatally by avoidance of stress and circumstances that increase endogenous methylamines and carefulness in contact with exogenous methylamines. Fetuses and babies are removed from environments with excessive responses of endogenous methylamines or they are not removed to such environments.

### BACKGROUND

Antidepressant drug therapies of depression existing now have been developed based on sedative and hypnotic effects of potentially antidpressant physical and chemical exposures in mature experimental animals. The number of clinical and experimental investigations that have been performed to examine antidepressant efficacy of potential and known antidepressant therapies such as drugs and electroconvulsive therapy in patients during adulthood and in mature experimental animals is innumerable. The approach has been invalid if considering the etiology, pathogenesis and pathology of depression and closely related disorders because the sedative and hypnotic properties of these potential antidepressant therapies were tested in patients and experimental animals during an ontogenetic phase when pathophysiology and pathology of vigilance and sleep already interacts with the pathogenesis, pathophysiology and pathology of hyperactivity, depression, excessive intake of alcohol, and alcoholism. This serious weakness in the theory of pathogenesis, pathophysiology and pathology of depression and the closely related disorders of hyperactivity in attention deficit disorder of children and excessive alcohol intake and alcoholism in adults and in investigations on the mechanism of antidepressant action is the reason for the ineffectiveness of the therapies available now for hyperactivity, depression, excessive intake of alcohol, and alcoholism which therapies are, though, predominantly selective in altering the function of monoamines in the brain and body, and therefore cause only few side effects. The relationship, which is probably causal, between early developmental function of monoamine neurons and their effects on postsynaptic tissues, both synaptically and hormonally, has been difficult to find because in human beings the incidence and prevalence of attention deficit disorder with hyperactivity, depression, increased voluntary intake of alcohol, and alcoholism requiring clinical attention is related to other common diseases with already an alteration in brain and body monoaminergic functions including cardiovascular (Acta Med. Scand. Suppl. 660: 69-83, 1982), and respiratory alterations such as snoring (Acta Physiol. Scand. 123:2: 56A, P77, 1985) and disorders of nutrition including alcoholism, autoimmune and collagen diseases, certain tumors, common neurologic diseases such as Alzheimer's disease and stroke, brain contusions, and Parkinson's disease, metabolic diseases, and side effects of commonly used medicines such as antihypertensives, analgesics (most notably morphine and other opiates), hypnotics and anxiolytics such as benzodiazepines (Alcoholism: Clin. and Exp. Res. 13: 2: 252, 1989). Consequent to the confounding effect of the pathological alterations inherent to the diseases or medications of them listed above, with the etiology, pathogenesis and pathology of hyperactivity, depression, and increased voluntary intake of alcohol, and alcoholism have remained unknown.

The simultaneous involvement of endogenous monoamines and exogenous biogenic amines (Liikennevilkku 5-6: 18-19, 1985; Med. Biol. 65: 97-104, 1987; Suomen maanpuolustuksen tieteellinen neuvottelukunta 2/A/87) or synthetic amines with methyl-amine-moieties in many common degenerative diseases in addition to hyperactivity, depression, increased voluntary intake of alcohol, and alcoholism renders clinical judgement even more important than earlier (Eur. J. Obstet. Gynecol. Reprod. Biol. 21: 283-291, 1986) when derivatives of methyl-amines are used in the treatment of attentions deficit disorder with hyperactivity, depression, excessive voluntary intake of alcohol, and alcoholism. The indications for treatment with methyl-amine derivatives will clearly depend on family history and results of laboratory examinations on the quality of sleep-waking cycles during the last two intrauterine and first four postnatal months because there exists probably also in humans, as shown to exist in laboratory rats, a genetic trait in sleep-waking cycles of offspring bred for increased voluntary intake of alcohol occurring during the second and third postnatal weeks (Acta Physiol. Scand. 154: 75-80, 1975) the nature of which has been later characterized in human beings in part of dopamine- and gamma-amino-butyric acid A-receptors. The early postnatal period is the only period of the ontogenesis of rats when circadian and seasonal rhythms of eg. illumination, temperature, and other environmental effects do not confound conclusions from altered monoaminergic functions caused by endogenous or ontogenetically more previous exogenous factors (e.g. Peptides 9: 487-491, 1988).

There are many animal models of depression which mimic the abnormal physiology and behavioural responsiveness of depressed human beings. One of these models is Porsolt's swim test (Pharmacol., Biochem. and Behav. 28: 367-369, 1987; Acta Physiol. Pharmacol. Latino Americana 37 (1): 164-166, 1987; Pharmacol. and Toxicol. 63: 57-61, 1988; Psychopharmacology 96: 353-359, 1988), and there are many other tests for detection of the effects of chemical and physical exposures on reactivity of experimental animals under potential or proven antidepressant and depressant exposures such as tests for measurements of startling (Acta Physiol. Scand. 132: 191-198, 1988), measurement of sleep-waking behavior after depressant exposure (Alcohol and Alcoholism 22: 3: 231-240, 1987; Pharmacol. and Toxicol. 64: 185-189, 1989; Peptides 11: 1-4, 1990), and measurement of voluntary intake of alcohol, open-field behavior, spontaneous alternation, and learned aversion (Developm. Brain Res. 15: 129-132, 1984; J. Neural. Transm. 70: 99-116, 1987; Alcohol and Alcoholism 22 (3): 231-240, 1987; and Suppl. 1: 339-343, 1987; Psychopharmacology 96: 353-359, 1988). Other tests include avoidance learning, chock-induced aggression, male and female sexual and reproductive behaviors of experimental animals which are related to antidepressant efficacy also in depressed human patients.

Monoamines, brain monoamines particularly, are directly or in the case of some other antidepressant treatments such as treatments with electric chock, with intensive lighting or with gamma-amino-butyric acid-ergic drugs, affected, and have, therefore, been considered to participate in the pathogenesis, pathophysiology, pathology, and symptoms of depression. The reasoning is, nevertheless, seriously confounded by the high incidence and prevalence of depression and abnormally increased voluntary alcohol intake in those persons who also suffer from other diseases which themselves alter monoaminergic functions in the brain and body.

The research and experimental therapeutic approach targeted at brain and body monoamines during adulthood would not a priori be capable of disclosing new understanding of the intrinsic nature such as the etiology, pathogenesis, and pathology of attention deficit disorder with hyperactivity, depression, excessive voluntary intake of alcohol, and alcoholism but could only be capable of disclosing the level of responsiveness to therapies such as drug treatments targeted at the symptoms of these world-wide prevalent and disabling diseases and conditions when applied to treat human beings during adulthood and experimental animals at a sexually and reproductively mature age.

Ontogenetically, both humans and animals spend the largest proportion of life in sleep during early development. This proportion is as large as three fourths during the ontogenetic period when monoamine neurons begin to be functional relative to their roles in neurotransmissions in the brain and begin to exert monoaminergic effects on tissues. It is noteworthy that reserpine, which depletes monoamine neurons from their neurotransmitter, and was earlier commonly used as an antihypertensive treatment of human patients with high blood pressure, caused clinical depression in as many as one fourth of the treated patients. Taken together these two facts I conclude that, theoretically calculating, the physiologic regulations of sleep-waking cycles would be lost by treatment with reserpine as a consequence of a loss of monoamines in the monoamine neurons resulting in a release of waking behavior from physiologic regulation manifested either as a depression or as a closely related condition or disease such as hyperactivity in children, excessive voluntary intake of alcohol, and alcoholism or depression in adults in every patient that presumably had depletion of monoamines during treatment with reserpine. During early ontogenetic development in experimental animals and presumably also in human beings (during the last two intrauterine and the first four postnatal months) monoamine neurons already exist and even their postsynaptic monoaminergic receptors in the brain and other target tissues already exist (Pharmacol. Rev. 43 (4): 553-561, 1991) but the monoamine neurons are empty of monoamines thus closely resembling the common side effect of the old practice of antihypertensive treatment with reserpine.

Therefore, it was logical to hypothesize that the onset of the pathogenesis of attention deficit disorder with hyperactivity, depression, excessive voluntary intake of alcohol, and alcoholism, and possibly even the etiology of these three closely related conditions and diseases (Duodecim 102: 17-25, 1986; Publicaciones del instituto de teoria, organizacion de la investigacion e historia de la ciencia no 5: 99-101, 1987; Acta Physiol. Scand. 154: 75-80, 1995) occurs during early ontogenetic development. An understanding of the nature of depression, and its major symptoms and that of related conditions and diseases, especially that of increased voluntary intake of alcohol and alcoholism in men, and hyperactivity in prepubertal children, are important for the formation of appropriate and effective concepts and strategies for the prevention and treatment of these two prevalent and disabling conditions and diseases more common in men than in women as well as in the related condition and disease of depression which is more common in women than in men.

In human beings, during prepuberty, which is Tanner M2 P2 in girls of 8-12 years of age, and G2 P2 in boys of 9-14 years of age, developmental disorders of epinephrine, dopamine, norepinephrine, and serotonin (J. Neural Transm. Gen. Sect. 102: 139-148, 1995) can be manifested as an attention deficit disorder with hyperactivity. This neuropsychiatric developmental disorder is common with a prevalence of 5-10 percent in children of school-age (Duodecim 102: 17-25, 1986). Boys who have been diagnosed as having attention deficit disorder with hyperactivity during prepuberty have also a very high carry-over of the underlying defect in monoaminergic neurotransmissions to later life manifesting then as a very high incidence and prevalence of alcoholism (J. Neural Transm. Gen. Sect. 102: 139-148, 1995). Thus, my invention can also be used as an animal model of the symptom of hyperactivity in the attention deficit disorder of children This model is related and close to a valid animal model of attention deficit disorder of children only to a limited extent because the most important symptoms are cognitive such as spelling and reading disabilities (Duodecim 102: 17-25, 1986) in addition to an inappropriate inattention for the existence of which increased ambulation and abnormally altered changes of habituation in the open-field in rats at the age of one month (J. Neural Transm. 79: 99-116, 1987; Psychopharmacology 96: 353-359, 1988) and at the age of two months (Developm. Brain Res. 15: 129-132, 1984) together with impaired habituation in a T-maze at the age of two months (Psychopharmacology 96: 353-359, 1988) and also together with a change of the normally stimulatory action of alcohol to a calming one at the age of two months (Developm. Brain Res. 15: 129-132, 1984) can for obvious reasons give only partial behavioral support. Thus, at the present state of knowledge, the animal model can be considered valid only for the symptom of hyperactivity in attention deficit disorder of children. The increased reporting of bizarre dreaming and nightmares both in children suffering from attention deficit disorder and in adults suffering from depression, excessive voluntary intake of alcohol, or alcoholism can in human beings be caused by an alteration in beta-adrenergic neurotransmission in the limbic forebrain and cerebellum, both brain areas of which contain physiologically a high density of beta-adrenoceptor binding sites relative to other brain areas. After early postnatal exposure to propranolol rats exhibit at the age of five months an increased content of norepinephrine in the limbic forebrain and cerebellum indicating the persistence of an alteration of beta-adrenergic neurotransmission in rats at this mature age. The lack of effect of propranolol exposure during early development on concentrations of serotonin and 5-HIAA indicates that the possible direct effects of early postnatal exposure to propranolol which could have caused long-lasting or permanent changes in serotonin receptors, suggested earlier to explain part of the bizarre dreaming on the grounds of effects of propranolol and pindolol on sleep-waking cycles in mature cats (Med. Biol. 56: 138-143, 1978; Sleep 1978, S. Karger, Basel, p. 485-488, 1980), are less important than direct beta-adrenergic effects in this model.

There is also suggestive evidence from experimental animals that boys of alcoholic fathers can develop alcoholism during later life because during early development there has been a condition which according to my invention occurred during the last two intrauterine and the first four postnatal months suggesting a genetic trait for development of alcoholism. Importantly, to this trait of increased voluntary alcohol consumption during later life is related an increased amount of active sleep in the sleep-waking cycles in rats during the second and third postnatal weeks of a certain rat strain, namely Alko Alcohol (AA) strain, selectively bred for increased voluntary intake of alcohol (Acta Physiol-Scand. 154: 75-80, 1995).

In order to examine the importance of the physiologic regulation of early developmental sleep-waking cycles for behavior during later life it would not have been possible to affect early postnatal active sleep by increasing or decreasing its relative proportion of time by changing the movements of pendulums on which mother rats with their offspring had been placed (for ref. see Duodecim 102: 17-25, 1986). Decreasing active sleep by restricting the relaxation of skeletal muscles in the necks of experimental animals by using a cuff pedestal technique (Physiol and Behav 32: 945-947, 1984) is not applicable to developing animals. It was necessary to interfere with early physiologic regulations of sleep-waking cycles by pharmacologic means.

Physiology of Sleep Revisited

A combined blockade of alpha-1 and alpha-2 adrenoceptors by phentolamine had been reported to increase active sleep in kittens (Acta Physiol. Scand. 109: 14A, 1980; Proceedings of the fifth congress of the European sleep research society, Amsterdam, the Netherlands, 1980) and in mature cats (Acta Physiol Scand 100: 488-490, 1980). The relative importance of different types of adrenoceptors, especially of those of alpha-1 and alpha-2 adrenoceptors, in physiologic regulations of sleep-waking cycles had and have been difficult to establish (Sleep 1982, ed. W. P. Koella, S. Karger, Basel, Switzerland, pp. 264-266, and pp. 270-272; Sleep: Neurotransmitters and neuromodulators, eds. A. Wauquier, J. M. Gaillard, J. M. Monti, and M. Radulovacki, Raven Press, New York, N.Y., U.S.A., 1985, pp. 69-77).

In kittens pretreated with a toxin of norepinephrine neurons, 6-hydroxy-dopamine (6-OHDA), clonidine, an alpha-2 adrenoceptor agonist, at a dose of 0.01 mg/kg of body weight intraperitoneally (i.p.), which suppresses paradoxical sleep (REM sleep) in mature cats (Life Sci 21(8): 1059-1066, 1977), suppresses active sleep also during early postnatal development, i.e. when cats are at two weeks and one month of postnatal age, and that prazosin, a pure alpha-1 adrenoceptor antagonist, at a dose of 0.5 mg/kg i.p., which in mature cats increases paradoxical sleep (Eur J Pharmacol 65: 417-420, 1980), does not affect active sleep in kittens which had been pretreated with 6-OHDA, at two weeks and one month of postnatal age. A hypersensitivity state of alpha-2 but not of that of alpha-1 adrenoceptors in monoaminergic connections regulating physiologically sleep-waking cycles in kittens was proposed (Alpha-1 and alpha-2 adrenergic modulation of vigilance and sleep (1985) Sleep: Neurotransmitters and neuromodulators, Raven Press, New York, N.Y., U.S.A., pp. 69-77).

The functional optimum of alpha-1 and alpha-2 adrenergic effects for different stages of waking and sleep in both kittens and mature cats can be concluded to depend on the amount (density) and the sensitivity of alpha-1 and alpha-2 adrenoceptors in different anatomic locations of tissues receiving noradrenergic or adrenergic pathways and innervations the sensitivity of which receptors appears not to change physiologically in alpha-1 adrenoceptors but appears to change physiologically in regulations of sleep-waking cycles only in alpha-2 adrenoceptors, and also in beta-1 adrenoceptors (Academic dissertation: Ilkka Hilakivi: The role of alpha-1 and beta-1 adrenoceptors in the regulation of the sleep-waking cycle of the cat, publicly discussed by permission of the medical faculty of the university of Helsinki, at the department of physiology, on Jan. 18, 1984, at 12 o'clock noon, ISBN 951-99498-5-2, Helsingin yliopiston monistuspalvelu, painatusjaos, Helsinki, 1984). At least in mature cats alpha-1 and beta-1 adrenoceptors can be concluded to exert in physiologic regulations opposite effects on many of the stages of sleep-waking cycles of cats (Acta Physiol Scand 473: 62, 193, 1979; Neurosci Lett 5: S394, 1980; Neurosci Lett Suppl 7: S484, 1981).

The current theory of monoaminergic regulations of sleep-waking cycles in human beings and in animals was and still is based on the important functions of endogenous, in particular cerebral, monoamines (Med. Biol. 65: 97-104, 1987). Suppression of active sleep by chlor-imipramine or by physical means such as the pendulum technique during early development affects profoundly sexual behavior, in particular ejaculation behavior of males as demonstrated in laboratory rats, the weight of cerebral cortex, and the content of deoxyribonucleic acid (DNA) and proteins in the rat brain during later life (Brain Res. 204: 129-146, 1981; Developm. Brain Res. 7: 277-286, 1983; Developm Brain Res. 7: 102-105, 1983). Inhibitors of neuronal reuptake of monoamines interact with alpha-1, alpha-2, beta-1, and beta-2 adrenergic and noradrenergic drugs on physiologic regulations of the stages of sleep-waking cycles in mature cats (Fourth international congress of sleep research, Association for the psychophysiological study of sleep (APSS), Bologna, Italy, Jul. 18-22, 1983; Pharmacol and Toxicol 60: 161-166, 1987). When administered to rats during their early ontogenetic development, monoamine reuptake inhibiting drugs exert long-lasting effects as was first shown to occur with chlor-imipramine on alcohol related behavior, on behavior during later life (Fourth international congress of sleep research, association for the psychophysiological study of sleep (APSS), Bologna, Italy, Jul. 19-22, 1983; Developm Brain Res 15: 129-132, 1984). Treatment of human mothers during pregnancy and during the period of the lactation of the offspring was warned to have long-lasting or possibly permanent effects on the human offspring (Eur. J. Obst. Gynaecol. and Reprod. Biol. 21: 283-291, 1986).

Although it was known that active sleep (also called rapid eye movement or REM sleep) occupies a large proportion of time during late fetal and early postnatal development there existed no other feasible method than observation of behavior to examine whether early developmental sleep was quiet or active and what was the proportion of early life-time spent in waking behavior. This was and is important because these three states have physiologically different monoaminergic regulations (Pharmacol. and Toxicol. 60: 161-166, 1987; Med. Biol. 65: 97-104, 1987). Quiet sleep is associated with synchronized slow, high amplitude, electric waves and active sleep with desynchronized fast, low amplitude, waves in the neuronal activity of the brain of mature human beings and animals. Increased deep slow wave sleep and suppressed REM sleep are associated with the effects of citalopram, a preferential inhibitor of serotonin reuptake, having a methyl-1 amine group in its chemical formula attached to a propyl-chain and having two additional methyl-groups attached (Adv in Physiol Sci Vol 1: Regulatory functions of the CNS, Principles of Motion and Organization, eds. J. Szentagothai, M. Palkovits, J. Hamori, Akademiai Kiado, pp. 329-332, 1980; Pharmacol and Toxicol 60: 161-166, 1987; Arch int Pharmacodyn 225: 317-329, 1977). Prindamine, a preferential inhibitor of norepinephrine reuptake, increases aroused waking, lengthenes the latency for REM sleep, and decreases the proportion of REM sleep.

Because electric activity of the brain develops not earlier than during early ontogenetic development it was and is obligatory to rely on and use other criteria for the classification of physiological and behavioral states relative to time during early ontogenesis. During early ontogenesis only observation of behavior was available for scientific use in experimental animals. Therefore, we developed a new application for a at that time relatively new method to detect movements in the head, body and extremities, and respiratory movements in newborn rats (Behav. Brain Res. 19: 241-248, 1986).

Since the movement sensitive method that had recently been developed was noninvasive we applied it for classification of the stages of wakefulness and sleep in human beings. Standardization of intraindividual, interindividual, and environmental circumstances is difficult when planning reliable vigilance and sleep experiments on human beings. There already existed epidemiologic results on sleep habits and sleep disorders of young men before and during their military service in the Finnish defence forces (Sleep 1980, S. Karger, Basel, 1981, pp. 383-385). We examined daytime vigilance and night sleep of young men during military service by using both conventional electrophysiologic methods in the laboratory and by using the relatively new movement sensitive method in the barracks (Acta Physiol. Scand. 123 (2): 56A, P77, 1985; Electroenceph. clin. Neurophysiol. 64: 89P, 1985). After the basic principles for analysing and scoring recordings of human beings with the then relatively new movement sensitive methods were introduced (Annal univ. Turkuensis Med-Odont. 26: 1-130, 1987) normative data on night sleep of healthy men aged 20±0.5 years were obtained based on the records on tape of nocturnal movements of the above mentioned particular men sleeping in beds in the barracks of a transportation battalion. The men had had either no complaints about daytime vigilance or night sleep before military service or had had a complaint of excessive daytime sleepiness, long night sleep, or snoring, or combinations of these complaints before military service. The records on tape were scored and analyzed according to these new principles (Acta Neurol Scand 86: 616-621, 1992). A personality test was validated to predict safety of operation of motor vehicles in a prospective follow-up of 11 months during the military service of men in the transportation battalion (Accid. Anal. and Prev. 21 (5): 413-418, 1989).

New Branch of Monoamine Research

Surprisingly, the possible significance of early ontogenetic functional development of brain and body monoamine neurons and neuronal circuitry had not been investigated previously in relation to the reportedly altered function of monoaminergic neurotransmissions in attention deficit disorder with hyperactivity during childhood, and depressive episodes, bouts of alcohol drinking or chronic alcoholism at adulthood in the kind of paradigma that was now available.

Early ontogenetic development of epinephrine, dopamine, noradrenaline, and serotonin neurons in experimental animals, most notably in rats, was in the eighties quite well-known in anatomic, biochemical, physiologic, and pharmacologic terms. Activity-inactivity cycles begin early in utero, which beginning is followed by a continuous and gradual development throughout the late intrauterine phase, early postnatal period, childhood, adolescence, maturity, and old age, towards death. Degenerative changes emerge during different phases of ontogenesis increasing during old age towards death. Already during mature adult age and especially during old age pathogenesis, pathophysiology, and pathology begin to interact with physiologic life and with advancing age increasingly also with pathophysiology and pathology of vigilance and sleep. There is evidence from cardiovascular physiology and diseases (Acta Med. Scand. Suppl. 660: 69-83, 1982). Pathophysiology of respiratory functions possibly begins as early as around 20 years of age as become exposed by symptoms such as snoring and by alterations in laboratory investigations. It is possible for example to find correlations with symptoms of vigilance and sleep disorders with quality and quantity of nocturnal movements in bed (Acta Physiol Scand 123 (2): 56A, P77, 1985). Primary pathology and primary diseases of vigilance and sleep such as narcolepsy also begin during adolescence or early adulthood (Narkolepsiaan liittyvän katapleksian patofysiologia ja hoito, 1983, Uni ja elimelliset unihäiriöt, eds. H. Lang, B. Falck and J. Hasan, Kiasma, Parainen, pp. 148-156). Strictly theoretically, therefore, the only valid period of life to search for a possible connection between physiology, pathophysiology, and pathology of sleep-waking cycles with those of attention deficit disorder with hyperactivity during childhood, depression and its major symptoms in adulthood, and excessive voluntary intake of alcohol and alcoholism in adulthood, is the early ontogenetic development.

Monoamine neurons develop their significance as mediators of neurotransmissions in human beings during the last two intrauterine and first four postnatal months. In rats these neurons develop their physiologic significance in neurotransmissions during the second and third postnatal weeks.

Attention deficit disorder with hyperactivity, depression and its major symptoms, and increased voluntary intake of alcohol, and alcoholism share during early ontogenetic development, in addition to the functional development of monoaminergic neurotransmissions, development of extramonoamine neuronal networks and pathways such as those of endogenous morphines and endorphins (Peptides 11: 1-4, 1990), those of excitatory neurotransmitters glutamate and aspartate as well as the minor metabolite of the dominantly inhibitory monoamine neurotransmitter serotonin (Pharmacol and Toxicol 64: 185-189, 1989), and those of neuropeptides such as the nonapeptide arginine-vasotocin (Peptides 9: 487-491, 1988) which, however, now, are beyond the scope of this invention. It is noteworthy, though, that arginine-vasotocin is a candidate substance for use in the induction of early postnatal active sleep during the last two intrauterine and first four postnatal months in human babies to treat psychiatric disorders connected pathophysiologically with physiologic or pathophysiologic regulations of sleep-waking cycles during early ontogenetic development such as are inferred to be associated with attention deficit disorder with hyperactivity, depression, excessive voluntary intake of alcohol, and alcoholism. This is reasonable because arginine-vasotocin was reported to induce rapid eye movement sleep in prepubertal boys and in patients with narcolepsy or symptomatic hypersomnia (ref in Peptides 9: 487-491, 1988). Thus, the emotional well-being is increased, daytime vigilance, and capabilities for performance such as operation of motor vehicles can be expected to improve (Accid Anal and Prev 21 (5): 413-418, 1989; Sotilasterveydenhuolto—Military Health Care, ed. K. Koskenvuo, 3. print, 1996, pp. 776-781). It is also possible to diagnose disruption of physiologic cycles of waking, quiet state and active sleep and in such cases to verify the efficacy of treatments using chemical or physical approaches during the first four postnatal months and possibly also during the last two intrauterine months of human babies by noninvasive methods to detect treatment-induced effects on waking, quiet state, and active sleep.

This invention is limited to physical, chemical, and biological exposures (Duodecim 102: 17-25, 1986; Suomen maanpuolustuksen tieteellinen neuvottelukunta, Helsinki, Finland, report 2/A/87) that are administered to alter early ontogenetic (in human beings two last intrauterine and first four postnatal months) monoaminergic dysfunctions that have been detected with sufficient validity and reliability to be so, indirectly in measurements of cycles of waking, quiet state, and active sleep of the baby and with anamnestic information, together with the results of physical and laboratory investigations performed on the baby, either by affecting monoamine (epinephrine, dopamine, norepinephrine and serotonin) neurons directly or by affecting them indirectly by targeting their postsynaptic receptors on neuronal and other target tissues which receive monoaminergic signals synaptically or, in certain anatomic locations and functions, hormonally, and alter their physical, chemical, and physiologic functions in metabolic, electromagnetic, and pharmacologic terms.

The introduced animal model can be used to test the effects of environmental physical, chemical, and biologic exposures that interact directly or indirectly with methyl-amine-moieties at receptors of endogenous monoamine neurotransmitters. These exposures interfere with physiologic interactions between neurotransmitters and their receptors, synthesis of neurotransmitters from their precursors, storage and release processes, and catabolism during the last two intrauterine and first four postnatal months of human babies. These exposures may happen during submissions to medical examinations, procedures, treatments, and health care, nutrition of babies and their mothers, or during the period when mothers during late pregnancy and the first four months of lactation are being employed under risky circumstances as part of their work engagements. The testing of the nature of the exposures mentioned above can be performed on otherwise intact healthy laboratory rats aged from one to three weeks. The rats are kept exposed to the same physical, chemical, and biologic environmental risks as human babies with their mothers. Health risks will be assessed in the same way as the effects of antidepressant drugs given during the second and third postnatal weeks on physiology and behavior during later life were assessed in previous experiments: It is possible to measure behavior during later life selecting tests for special purposes (Developm. Brain Res. 15: 129-132, 1984; Pharmacol., Biochem. and Behav. 28: 367-369, 1987; J. Neural Transm. 70: 99-116, 1987; Alcohol and Alcoholism Suppl. 1: 339-343, 1987; Acta Physiol. Pharmacol. Latino Americana 37 (1): 164-166, 1987; Acta Physiol. Scand. 132: 191-198, 1988; Pharmacol. and Toxicol. 63: 57-61, 1988; Psychopharmacology 96: 353-359, 1988; Eur. J. Pharmacol. 271: 223-226, 1994). It is also possible to select measurements of samples of sleep-waking behavior during the exposure (Behav. Brain Res. 19: 241-248, 1986). It is possible to select measurements of monoamines and their metabolites after risky exposures (J. Neural Transm. 70: 99-116, 1987; Alcohol and Alcoholism Suppl. 1: 339-1343, 1987; Psychopharmacology 96: 353-359, 1988; J. Neural Transm. Gen. Sect. 102: 139-148, 1995; Pol. J. Pharmacol. 49: 17-20, 1997; Pol. J. Pharmacol. 51: 311-316, 1999). If the exposures of mothers and their babies have been evaluated to be dangerous for health it is even possible to perform treatment trials in experimental animals with treatments targeted selectively (e.g. GABA-B receptors or beta-adrenoceptors, Pharmacol. and Toxicol. 63: 57-61, 1988).

There is a very strong burst of secretion of especially epinephrine and also other catecholamines from adrenal medullas into the blood circulation during birth causing their distribution into the blood stream and then into tissues overall an unphysiologically high concentration of methylated endogenous amines, mainly epinephrine. The small, intensely fluorescent granule-containing cells in the sympathetic ganglions are directly relevant contributing to the sympathetic reaction of the body during the enormous birth stress of early human and other mammalian life (O. Eränkö and L. Eränkö, in: Histochemistry of nervous transmission, ed. O. Eränkö, Elsevier Publishing Company, Amsterdam, the Netherlands, 1971, pp. 39-51). The blood-brain barrier for physiologically active monoamine neurotransmitters is poorly functional during late pregnancy, birth and very early postnatal development. Therefore, catecholamines released from adrenal medullas have access and reach also neuronal target tissues inside the meninges which during later life confine the central nervous system as a more separate compartment with respect to monoaminergic neurotransmitter functions than before and during birth. Sympathetic neuronal response to stress (norepinephrine, acetylcholine in certain functions) contributes little to the concentrations of catecholamines and their metabolites in the blood stream. In the passage and transports through the blood-central nervous tissue barrier vesicular transport through capillary endothelium, pial and intraparenchymal arterioles to perivascular spaces containing interstitial fluid, have significant functions; also migrating cells exerting specific functions such as immune cells, and cells of ependyma, affect movements of monoamines, their precursors and metabolites towards and from the brain ventricles, and also through the blood-retina barrier. The noradrenergic and acetylcholinergic innervations of blood vessels of the central nervous system are involved in passage and transport processes through the blood-central nervous tissue barriers. There is additionally active uptake into the barrier of precursors of monoamine neurotransmitters from the blood, their decarboxylation and oxidation, diffusion, facilitated transport by coupling to transport of other substances through the barrier, and active transport consuming energy, thus resulting in changes of concentrations of monoamines, their precursors and metabolites in the central nervous system (Pharmacol. Rev. 28 (4): 275-348, 1977). During early development the movement of amines through the blood-central nervous tissue-barrier results in an excess of the N-methylated monoamine neurotransmitter epinephrine relative to the non-N-methylated neurotransmitters dopamine, norepinephrine, and serotonin, and the non-N-methylated diamine histamine. In some babies the blood-central nervous tissue barrier may be exceptionally permeable to epinephrine and structurally related methylated amines.

During the last two intrauterine and first four postnatal months in human babies it is beneficial with respect to the risk of the onset of the pathogenesis of attention deficit disorder with hyperactivity in childhood, depression, excessive voluntary intake of alcohol, and alcoholism in adulthood to suppress or reduce excessive release of epinephrine from the adrenal medullas into the blood stream and its distribution inside the brain, medulla oblongata, and tissues of the body. This is accomplished by reducing or preventing the synthesis of epinephrine from norepinephrine in the adrenal medullas by inhibiting the endogenous enzyme phenyl-ethanol-amine-N-methyl transferase (PNMT) localized in the adrenal medullas. Consequently, the excessive effects of epinephrine and other N-methylated amines as a result of metabolic processes in the brain and body are reduced or prevented. The suppression or reduction of the synthesis of epinephrine, though, must be accomplished before stressful events, most importantly before major diseases or risky exposures to chemical, physical, including major surgery, or biologic agents during late intrauterine life, during birth complicated because of obstetric problems, and during lactation by mothers under risky conditions happen to take place in order to be effective in reducing or preventing an excessive activation of the peripheral autonomous nervous system and most notably the excessive release of epinephrine into the blood stream.

The reduction or prevention of the onset of the pathogenesis of attention deficit disorder with hyperactivity in childhood, depression, excessive voluntary intake of alcohol, and alcoholism in adulthood must be targeted chemically and timed specifically. It is physiologic to render throes of child birth as gentle as possible and to avoid any stress of the baby and mother during the last two months of pregnancy and the first four months of lactation. Stress increases the probability of the onset of pathogenesis and pathophysiology of attention deficit disorder with hyperactivity, depression, excessive voluntary intake of alcohol, and alcoholism, and also the probability that they are manifested after a possible latent period during later life. If obstetricians and pediatricians are aware that a significant stressful event had happened such as a major accident, surgery, or disease episode, and the condition continues to prevail in the mother and fetus or baby, it is morally obligated to reduce or prevent the adverse effects of such unphysiologic stresses on later life by treatment. The treatment can be accomplished during the last two intrauterine and the first four postnatal months by giving to the fetus or newborn baby pharmacologic agents that pass the blood-central nervous tissue barrier and act there like endogenous dopamine, norepinephrine, and serotonin, which are the three endogenous monoamine neurotransmitters sharing an ethyl-amine-moiety, and pretreating the fetuses or newborn babies with a PNMT inhibitor which does not necessarily need to pass through the blood-central nervous system-barrier but can act mainly on the adrenal medullas to reduce the amount of epinephrine, and therefore also its N-methylated metabolites, in the blood and other tissues as responses to unphysiologically stressful stimuli. The drugs to be targeted at the pathogenesis and pathophysiology of attention deficit disorder with hyperactivity, depression, excessive voluntary intake of alcohol, and alcoholism can be N-substituted at the moiety of methyl-amine to be able to reach the same receptors as pathologically prevailing methyl-amines do and to compete with the pathologically prevailing methyl-amines and their metabolites or in other ways counteract the pathological consequences on neurotransmission of an excessive concentration of methyl-amines in the central nervous tissues which can include but does not necessarily include an excessive concentration of epinephrine which prevails and functions in physiologic concentrations as a neurotransmitter. Adrenergic circuitry and pathways in the central nervous system can also specifically malfunction and release excessive amounts of epinephrine from varicocities. During early ontogenesis (last two fetal months and first four postnatal months) there can be epinephrine available in the central nervous system as a result of immature functioning of the blood-central nervous system-barrier. The endogenous monoamine neurotransmitters dopamine and serotonin are pure ethyl-amines. The molecules of epinephrine and norepinephrine have in their ethyl-chains hydroxyl-moieties attached. These hydroxyl-moieties are necessary for granular storage in neurons. The three neuropsychiatric disorders hyperactivity during childhood, depression, excessive voluntary intake of alcohol, and alcoholism share partly same underlying neuronal functions as the physiologic regulations of sleep-waking cycles. Thus, it is possible that measurement of cycles of waking, quiet state, and active sleep during the last two fetal months and the first four postnatal months of human babies can add to diagnostics and be even used to verify the efficacy of treatment of the underlying pathogenesis and pathophysiology of the three neuropsychiatric disorders.

In the regulation of sleep-waking cycles of cats a higher level of beta-1 adrenergic activation relative to alpha-1 adrenergic activation in the brain facilitates activation of neurons underlying electroencephalographic, electromyographic, electro-oculographic, and behavioral aspects of paradoxical or rapid eye movement (REM) sleep, which corresponds with active sleep during development when phasic phenomena such as twitching of striated muscles are exaggerated compared with REM sleep during later life. This ratio is opposite to the ratio of a higher alpha-1 than of beta-1 level of activation correlated with and causally related to the electroencephalographic and behavioral criteria and signs of the waking state (Acta Physiol Scand Suppl 473: 62: 193, 1979; Helsingin yliopiston monistuspalvelu, painatusjaos, ISBN 951-99498-5-2, 58 pages, 1984). So, during early development the excessive amount of methylated amines would lead to an increased level of activation of beta-1 adrenergic connections relative to alpha-1 adrenergic connections in neuronal circuitry and pathways. Beta-1 adrenergic stimulation by prenalterol, indeed, increases the amount of paradoxical (REM) sleep in cats at mature age (Neurosci Lett Suppl 7: S484, 1981; Brain Res 277: 109-118, 1983). There are reports of bizarre dreaming and nightmares in patients receiving treatments with beta-adrenoceptor antagonists that have physiologically and cardiovascularly therapeutically proven to exert physiologically significant intrinsic beta-adrenoceptor agonist activity, most notably pindolol (Fifth Scand meeting on physiol and behav, Helsinki, Finland, 1977, p. 13; Med. Biol. 56: 138-143, 1978). A very low dose of pindolol (0.5 mg/kg of body weight but not 0.1 mg/kg of body weight, intraperitoneally, in cats increases the amount of REM sleep and also increases the amount of drowsy wakefulness during which hypnagogic and hypnapompic dreaming-like phenomena may occur thus possibly explaining or partly explaining the reports of bizarre dreaming by patients receiving treatments with this beta-adrenoceptor antagonist with intrinsic agonist activity, the amounts of deep slow wave sleep and paradoxical sleep are concurrently decreased (Med. Biol. 56: 138-143, 1978). The beta-adrenoceptor agonist isoproterenol that has in its structural formula three methyl-moieties in the N-position of the amine-moiety was later found to increase active sleep during ontogenetic development in a rat model (C. R. Acad. Sci. Paris 292 (III): 645-647, 1981; Eur. J. Pharmacol. 79: 257-264, 1982). Thus, for active sleep to happen during early development only blockade of postsynaptic alpha-adrenoceptors sustains a ratio of postsynaptic alpha-1 and beta-adrenergic level of activation which is physiologic for active and REM sleep. Excessive stimulation of postsynaptic alpha and beta-adrenoceptors leads to changes in neuronal activity arousing experimental animals in laboratory investigations if beta-1 adrenergic stimulation-induced activation of alpha-noradrenergic connections in neuronal circuitry and pathways leading to awakening up of the animals are not blocked by postsynaptic alpha-1 adrenoceptor antagonists (Eur. J. Pharmacol. 65: 417-420, 1980; Acta Physiol. Scand. 120: 363-372, 1984; Pharmacol., Biochem. and Behav. 24: 613-616, 1986; Med. Biol. 64: 355-360, 1986; Physiol. and Behav. 39: 551-553, 1987). Epinephrine has a higher affinity for beta-adrenoceptors and for alpha-2 adrenoceptors than for alpha-1 adrenoceptors which, therefore, is a dominantly noradrenergic receptor. Thus, the three neuropsychiatric conditions and diseases, hyperactivity during childhood, and depression and alcoholism during adulthood can be concluded to result from an onset of pathogenesis and pathophysiology caused by an excess of methylated amines changing the physiologic ratio of adrenergic alpha-2 and beta receptor stimulation relative to dominantly noradrenergic alpha-1 receptor stimulation in critical anatomic locations in the brain and body during the two last fetal months and the first four postnatal months of human beings concurrently disrupting cycles of waking, quiet state, and active sleep.

The etiology of the excess of N-methylated amines in the brain and body varies, and can be either inherent (genetic or prenatal epigenetic) manifesting only at the period of ontogenesis when monoamine neurons become physiologically functionally active in the brain and body, or exogenous.

In mature cats slow wave sleep is divided into light and deep slow wave sleep the latter of which is related to paradoxical (REM) sleep in terms of regulations of sleep-waking cycles (Brain Res 11: 347-356, 1968). Very interestingly, this functional linkage of the deep stage of slow wave sleep and REM sleep is switched off by citalopram, a selective inhibitor of serotonin reuptake, and devoid of direct effects on monoamine, histamine, and acetylcholine receptors. Citalopram increases the proportion of time cats are in deep slow wave sleep to as large as 50% of time, and decreases that of REM sleep to only 2% during 12 hours after its i.p. administration. Prindamine, a preferential inhibitor of norepinephrine reuptake and devoid of anticholinergic activity, on the contrary, causes a lengthening of REM sleep latency and a long-lasting and consistent decrease in proportion of time cats are in REM sleep to approximately 7 percent of during 16 hours after its i.p. administration without other significant effects on the stages of sleep-waking cycles of cats. Phentolamine but not either yohimbine, prazosin or propranolol restores significantly the relative proportions of stages of sleep-waking cycles towards baseline (Pharmacol. and Toxicol. 60: 161-166, 1987). The combination of prindamine and prazosin, which theoretically should increase the amount of synaptic norepinephrine available for noradrenergic pre- and postsynaptic alpha-2 and beta-receptors results in a true cat model of human insomnia. The synaptic functional state causing this insomnia in cats may be exaggerated by beta-adrenoceptor stimulation-mediated coupling to supersensitivity of alpha-2 adrenoceptors proven to happen in rat neuronal tissues (Science 207: 645-646, 1980). In addition to presynaptic alpha-2 adrenoceptors there exist postsynaptic populations of alpha-2 adrenoceptors, and can be inferred to be associated with the reduction of the duration of sleep to only one third of the physiologic quantity, and the change in its quality from deep slow wave and paradoxical (REM) sleep to light slow wave sleep with only very little deep slow wave and no REM sleep at all during the 16 hours after i.p. injection of this combination of drugs (Adv. Physiol. Sci. 1. Regulatory functions of the CNS. Motion and organization principles, eds. J. Szentagothai, M. Palkovits, and J. Hamori, Pergamon press, Akademiai Kiado, Budapest, Hungary, pp. 329-332; Pharmacol. and Toxicol. 60: 161-166, 1987). Insomnia is a common major symptom in both depression and abuse of alcohol.

Mature cats were deprived of paradoxical (REM) sleep by preventing them from relaxing their neck muscles by placing them on platforms surrounded by water and large enough for sitting on the platforms but not large enough for permitting the relaxation of neck muscles in the lying position on the platform. During recovery after a 72-hour sitting on the platforms (so called REM sleep deprivation procedure) the amount of REM sleep and the relative proportion of time cats are in REM sleep remain above normal for 24 hours. The high percentage of REM sleep during recovery after this procedure is caused by an increase in the number of sleep-waking cycles thus resulting in a larger proportion of time cats are in REM sleep relative to other stages of sleep-waking cycles. This procedure is known to activate several neural and hormonal adaptations in the brain and body that, interestingly, resemble responses of depressive patients and experimental animals to antidepressant treatments (Endocrinology 133 (1): 312-320, 1993; Trends in Neurosci 18 (1): 6-10, 1995). It is not mere adaptation to stress in neuronal, hormonal, immunological, or any specific terms because changing the size of the platforms to somewhat larger, which permits the relaxation of neck muscles does not change the responses in specific functions of the organism or individual to so large an extent. The natural purpose, teleologically, of the cycling of the states and stages of sleeping and waking is adaptation of the organism and individual to living in the universe. The changes in the overall behavior of the individual during time are called cycling of vigilance and different states and stages of sleep. There is an intraorganism, or intraindividual, training of physiology and behavior which in human beings also includes thinking and imaginary and which training of physiology and behavior happens during wakefulness in an interactive way with changes of the environment. The environment also trains overall physiology and behavior of the individual who is cycling in different stages of sleep, the most importantly between slow wave sleep and REM sleep. The simple procedure mentioned above trains physiology and behavior of the individual in a way that during this procedure the proportions of time in drowsy waking and light slow wave sleep are increased, and those of deep slow wave sleep and paradoxical (REM) sleep are decreased but that of aroused waking is not changed. After submission to this procedure there is a recovery phase during which the proportion of aroused waking is not changed but the proportions of deep slow wave sleep and paradoxical sleep are increased and those of drowsy waking and light slow wave sleep are decreased. These changes in sleep are causally related, directly or indirectly, with changes in monoaminergic neurotransmissions in the brain and body. If this overall behavior of the individual is trained in such a way as in the above mentioned procedure the adaptive capability of the individual is reduced also during aroused waking secondary to the effects of an altered function of monoaminergic neuronal circuitry and pathways in the brain and body thus causing training of the overall physiology and behavior of the individual towards an asynchrony with such operative states and stages of monoaminergic functions that are physiologically and psychologically healthy or normal for complex waking behavior trained continuously by intraindividual physiologic responses to interindividual and environmental stimuli such as inhibitions, demands, rewards, provocations, challenges, desires, and wardings off including sexual, reproductive, and other physiologic, psychologi, social, and environmental stimuli—with varying timing and duration as well as magnitude. It is reasonable to conclude that the quiet or drowsy stages of waking and different states and stages of sleep exert a physiologic and psychologic (mental) training effect on waking behavior the disturbance of which in the case of disrupted synchrony of monoaminergic functions in the brain and body in relation with the training effect of physico-chemical, psychologic, and social environment onto the brain and body may result in attention deficit dirorder with hyperactivity in childhood, depression, excessive voluntary intake of alcohol, and alcoholism in adulthood.

During the 24-hour recovery after sitting on a small platform physiologic states with fast low amplitude electrocorticogram, with rapid bursts of eye movements, bursts of electric activity in the lateral geniculate bodies of the thalami, and with relaxation of neck muscles accompanied with phasic switches in small muscles of the extremities (paradoxical sleep, REM sleep) were triggered more frequently (baseline 45±9 cycles, recovery 73±19 cycles). Phentolamine given at the beginning of recovery tended to increase the duration of the episodes cats were in REM sleep: baseline 4.6±0.4 min, REM sleep deprivation 5.4±1.8 min, REM sleep deprivation plus phentolamine 20 mg/kg i.p. 6.5±1.9 min, n.s., t-test after ANOVA. Phentolamine did not affect the number of REM episodes. Evidently, there is a physiologic upper limit for the magnitude of quantitative changes in the sleep-waking cycles already approached with the sole small platform intervention even without phentolamine administration. (Fifth Scandinavian meeting on physiology and behavior, Helsinki, Finland, 1977, p. 35; Physiol and Behav 39: 551-553, 1987). There, however, exist species differences. In rats the combined alpha-1 and alpha-2 adrenoceptor antagonist phentolamine increases the frequency of REM sleep episodes in sleep-waking cycles and not the duration of them (Pharmacol., Biochem. and Behav. 24: 613-616, 1986). The physiologic modes of regulatory functions also differ between cats and human beings. For example, phentolamine increases in cats, which have a very reactive sympathetic nervous system in the brain as well as in the body, considerably cerebral blood flow which in certain regions is even doubled but does not affect regional cerebral blood flow in human beings (Pharmacol. Rev. 28 (4): 275-348, 1977). The pure alpha-1 adrenoceptor antagonist prazosin increases paradoxical (REM) sleep in cats to a considerable extent (Eur. J. Pharmacol. 65: 417-420, 1980) which is considerably more than in primates (Physiol. Behav. 37: 199-202, 1986). In cats prazosin increases more the duration of REM sleep episodes than the frequency of them in the sleep-waking cycles indicating that the inhibition of alpha-1 receptor activation in the brain and body in which the constriction of resistance vessels reset cardiovascular tone causing effects on the brain through vasovagal physiologic reactions resulting in a stupor-like state (a serotoninergic system in parasympathetic afferents, In: Sleep 1982, Proceedings of the sixth European congress of sleep research, Zurich, Switzerland, 1982, ed. W. P. Koella, S. Karger, Basel, pp. 64-68). These effects of prazosin result together with an increased ratio of beta-(mainly beta-1) adrenergic relative to alpha-1 receptor mediated (mainly noradrenergic) level of activation in the brain and body manifesting in overall physiology and behavior of the individual as longer episodes of REM sleep. An excess of N-methylated amines in the brain and partly also in the body may happen either through exogenous impacts or as a pathogenetic and pathophysiologic primarily endogenous phenomenon during the early phase of functional development and physiologic activation of monoaminergic neurotransmissions which in human being is the period of the last two fetal and the first four postnatal months resulting in a manifestation of neuropsychiatric conditions and diseases at an ontogenetic period when the training effects on physiology, behavior, and mental contents of sexual, reproductive, social, and environmental interactions become more continuous, demanding, and obliging to the individual.

The common symptom of attention deficit disorder hyperactivity is a prepubertal condition and disease whereas depression, excessive voluntary intake of alcohol, and alcoholism are postpubertal and related with sexual and reproductive behaviors.

In laboratory rats that have been bred for a high level of voluntary intake of alcohol (Alko AA strain) the duration of sleep-waking cycles and that of active sleep episodes during the second and third postnatal weeks are longer than in ordinary laboratory rats such as Wistar rats (Acta Physiol. Scand. 154: 75-80, 1995). Laboratory rats that during their later life are to choose to voluntarily drink a plenty of alcohol exhibit longer episodes of active sleep during their early postnatal ontogenetic period than ordinary rats exhibit, in this particular investigation, Wistar rats. AA rats are susceptible during this ontogenetic period to signals from themselves and from the environment that lead to awakening up from sleep as a result of active sleep episodes lasting excessively long time instead of a smooth switching from somewhat shorter episodes of active sleep to quiet state or quiet sleep thus permitting the continuation of unconsciousness of the environment and a better recovery from the effects on physiology and psychology of waking preceding the time of falling asleep. When these kinds of sleep-waking cycles continue to happen during the second and third postnatal weeks there may supervene an asynchrony of monoaminergic neurotransmissions with the physical, chemical, physiologic and psychologic training effects of environment resulting in monoaminergic neurotransmissions and behavior during later life which are different from those of ordinary rats, in this case Wistar rats, under the same training circumstances.

The combined alpha-1 and alpha-2 adrenoceptor antagonist phentolamine and the pure alpha-1 antagonist prazosin increase in rats aged 34 months the percentage of paradoxical (REM) sleep, which corresponds with active sleep during early ontogenesis, between 4 and 12 hours after an i.p. administration (Pharmacol., Biochem. and Behav. 24: 613-616, 1986). This increase is more due to an increased frequency of REM sleep episodes than to a lengthening of the duration of them.

Gender differences are involved in monoaminergic regulations of physiology and behavior during later life. Gender differences in rats in cerebral monoamine structure and function manifest only when sexual and reproductive behaviors begin after first month of development (Pol. J. Pharmacol. 51: 311-316, 1999). These gender differences are evidently complex and versatile and have differing durations of ontogenetic latent periods before manifesting as differences in sexual and reproductive behavior between boys and girls resulting in a large gender difference also in the pathogenesis and pathophysiology of monoaminergic functions in the brain and partly in the body manifesting during adulthood as depression, which about 11 percent of women suffer compared with only 3-4 percent in men, and in incidences and prevalences of excessive voluntary intake of alcohol and alcoholism which are opposite to those of depression relative to gender.

For the prevention and for a therapeutic approach slowing down of the onset and alleviation of the magnitude of pathogenetic, pathophysiologic, and pathologic changes during the last two fetal and the first four postnatal months N-substitution of methyl-amine moiety is critical in order to compete with or block the adverse effects of N-methylated amines on monoaminergic neurotransmissions of pathologically in the brain and body prevailing N-methyl-amines primarily consequently to endogenous genetic or conceptual or prenatal epigenetic causes or primarily to exogenous causes. In the chemical structural formula of adrenaline after the carbon atom with methyl-amine moeties the next carbon atom forming the ethyl-amine structure is subject to further physiologic variation and is under versatile physiologic regulations caused by variations with differences in this part of the molecule in the monoamine uptake into, storage in, and release from neuronal granules such as are caused by the administration of alpha-methyl-dopa resulting in a synthesis of alpha-methyl-norepinephrine, associated also with changes in sleep-waking cycles, in cats (Electroencephal. clin. Neurophysiol. 43: 4: E283, p. 525). The benzene ring is not essential, and is replaced by an indole-structure in serotonin molecule and by an imidazolyl ring in histamine molecule. Both serotonin and histamine as well as acetylcholine, which is a quaternary amine, are interesting in relation with the physiologic regulation of sleep-waking cycles (Med. Biol. 65: 97-104, 1987). A property of moderate blockade of alpha-1 adrenoceptors is achieved by N-substituting the amino-moiety of epinephrine with a quinoline ring which itself is associated with the property of being capable of increasing paradoxical sleep, in cats (Eur. J. Pharmacol. 65: 417-420, 1980), thus resulting in a new molecule with both the alpha-1 andenoceptor antagonist and beta-1 adrenoceptor agonist property, and possibly also with a property of alpha-2 adrenoceptor antagonism. New chemical compounds that alleviate or cure symptom of hyperactivity in attention deficit disorder, symptoms of depression, excessive voluntary intake of alcohol, and alcoholism based on alterations in monoaminergic neurotransmissions and change monoaminergic neurotransmissions in a way that prevents, counteracts or alleviates the asynchrony of endogenous and exogenous effects on monoaminergic neurotransmissions in the brain and body so that sleep-waking cycles during the last two human fetal and the first four human postnatal months become such that they do not result in maladaptations of specific waking behaviors such as hyperactivity, depression, excessive voluntary intake of alcohol or alcoholism, as a result of overall physiologic and psychologic dysregulations of sleep-waking cycles during early ontogenetic development.
2. N-substitution with quinoline ring with methylamine-moiety of epinephrine to develop epinephrine-N-quinolines acting like endogenous non-methylated amines (dopamine, norepinephrine, serotonin) and inhibiting the effect of a pathogenetically and pathophysiologically caused excessive amount of N-methylated amines with adverse effects on health prevailing in the brain and body, and returning sleep-waking cycles physiologic by inducing morel physiologic-like regulations of sleep-waking cycles for preventing, counteracting, and alleviating the asynchrony between endogenous, intraindividual physiologic and psychologic effects and exogenous interindividual and environmental effects on specific waking behaviors including mental contents and thinking, resulting in pure agonism of beta-1 adrenoceptors, and in the same molecule, pure antagonism of alpha-1 receptors; a moderate additional property of alpha-2 adrenoceptor blockade is possible resulting in a combination of the property of alpha-1 and alpha-2 adrenoceptor blockade and in an increased ratio of postsynaptic beta-1/alpha-1 adrenoceptor activation, for making, offering for sale, selling, marketing, using, and importing in order to treat during the last two intrauterine and the first four postnatal months the symptoms, mainly that of hyperactivity, in attention deficit disorder during childhood, depression, excessive voluntary intake of alcohol, and alcoholism in adulthood,
3. the making, offering for sale, selling, marketing, using, and importing of phenyl-ethanol-amine-N-methyl-transferase (PNMT)-inhibitors not necessarily passing through the blood-central nervous system-barrier or if so acting on adrenergic neurotransmissions in the brain stem of human fetuses during the last two intrauterine months and of human babies during the first four postnatal months to induce a reduction in the synthesis and storage of epinephrine available for release into the blood stream from the adrenal medullas and from epinephrine neurons to adrenergic synapses with dominantly alpha-2 and beta-1 adrenoceptors, and with a lesser potency, on alpha-1 receptors that are dominantly noradrenergic receptors, and on beta-2 receptors, for use solely, or in combination with the new molecules, as claimed in the claim 2, with properties of both alpha-1 adrenoceptor antagonism and beta-1 adrenoceptor agonism in the same molecule, and possibly additionally a property of alpha-2 adrenoceptor antagonism resulting indirectly in a higher ratio of activation between postsynaptic beta-1 and alpha-1 adrenoceptors, or in combination with both an already existing alpha-1 adrenoceptor antagonist drug and an already existing beta-1 adrenoceptor agonist drug, the alpha-1 adrenoceptor antagonist possibly also having a property to block alpha-2 adrenoceptors, to treat symptoms, mainly the symptom of hyperactivity, in attention deficit disorder during childhood, depression, excessive voluntary intake of alcohol, and alcoholism, and
4. using by administering in combinations of already existing pure beta-1 adrenoceptor agonists with already existing pure alpha-1 adrenoceptor antagonists with or without the additional property of alpha-2 adrenoceptor antagonism, which indirectly, by increasing the release of catecholamine neurotransmitters, increases the activation of postsynaptic beta-1 adrenoceptors, pre- and postsynaptic alpha-2 adrenoceptors, beta-2 adrenoceptors, and also, though less potently, alpha-1 adrenoceptors, for the treatment of hyperactivity of attention deficit disorder during childhood, depression, excessive intake of alcohol, and alcoholism during adulthood, by administering these combinations of already existing drugs during the last two intrauterine months to human fetuses and during the first four postnatal months to human babies.

1. Results of animal experiments proving a relationship between: ontogenetically early sleep-wake behaviour and neurobehavioural disorders during later life indicate examination of ontogenetically early sleep-wake behaviour in human beings. Under physical and chemical, atomic, and/or biologic exposures to agents which can during early ontogenesis, be administered to rats at dosages and conditions simulating environmental conditions, not different from the conditions of pregnant human mothers and their newborn babies who are being planned to be exposed or are bound to be exposed to the very same environmental conditions, are tested the effects of procedures intended for prophylactic use during early ontogenesis to improve neurobehavioural disorders during later life—otherwise resulting from the lack of sufficient physiologic early ontogenetic sleep, especially active sleep on the forebrain—such as childhood hyperactivity, adulthood depression, amphetamine, cocaine, ketamine, phencyclidine, and opioid dependence, and alcohol abuse and alcoholism, and states of hyper-S-adenosylhomocystenemia such as chronic renal failure or Alzheimer's disease, by using physical, chemical, physiologic, biochemical and behavioural tests measuring the effects caused by changes in endogenous methylamines. The fetuses and babies are not moved to environments with changes in endogenous methyl-amines or they are moved away. The fetuses during the last two intrauterine months and babies during the first four months after birth can thus be given prophylaxis by awareness and carefulness with contact, and avoidance when possible.

2. In a multitude of experiments on monoamine transmitter pathophysiology during early development of childhood hyperactivity, adult depression, amphetamine, cocaine, ketamine, phencyclidine, and opioid dependance, alcohol abuse, and alcoholism, and states of S-adenosyl-homocystenemia such as chronic renal failure and megaloblastic anemia or Alzheimer's disease a model is devised. A natural stimulus or force including all atomic, biologic, and physical-chemical exposures either through physical-chemical, gravitational, electrical or magnetic routes can increase the amount of methylamines in the brain. Technical features during the last two intrauterine months and the first four postnatal months can show—i.e. in electromagnetic recordings of cerebral neuronal activity sensing and integrating inner and outer universe, in movements of eyes and/or electromagnetic activity of the retinas and/or extraocular eye muscles, in electromagnetic activities of striated facial, nuchal or other skeletal muscles, and activation levels of movements measured by probes such as piezoceramic and/or static charge sensitive ones (dynamic and static, pressure and weak forces, gravity, performed movements), or in biochemical tests,—changes which would indicate abnormalities characterized typically by an altered level of active or rapid eye movement sleep and other deviations of normal sleep-wake behaviour. Early prophylaxis and therapy can be achieved during the last two intrauterine and the first four postnatal months by physical or chemical or biologic means including radiation activating of molecules like in this claim and the claims as follows. Physiologically during early ontogenesis active sleep stimulates the forebrain and the functional maturation, development, and growth of the forebrain in mood and affect characteristically altered dysregulation through ascending monoaminergic pathways from the brain stem which can in disturbances of sleep-wake behaviour during early ontogenesis under the influence of excessive amount of methylamines in the brain be achieved by therapy targeted at monoamine neurotransmitter pathways. Details of examinations to reveale the effects on physiology and behaviour of the specificity and potency of any effect known to affect monoaminergic neuronal and hormonal transmissions are intended to be used to improve by prophylaxis or treatment with drugs during early ontogenesis the symptoms of later-life neurobehavioural disorders such as childhood hyperactivity, adulthood depression, amphetamine, cocaine, ketamine, phencyclidine and opioid dependence, and alcohol abuse and alcoholism, and states of S-adenosyl-homocystenemia and Alzheimer's disease associated with methylamine harms and injuries. The present invention comprises a method of using already existing electro-magneto-encephalographic, electro-magneto-oculographic, and electro-magnetomyographic as well as actographic recordings to detect during the two last intrauterine and the first four postnatal months of human ontogeny a reduced amount of active sleep and other sleep-wake abnormalities in association with the clinical diagnosis of an abnormal state of sleep-wake behaviour of the baby, and often of mother who is often depressive or at risk of developing depression, and, consequently, the underlying pathophysiology in the monoaminergic regulations of sleep-wake behaviour and the cause for later life behavioural abnormal conditions and diseases as said, in order to render possible the clinical pharmacologic and therapeutic use of compositions of matter by characteristically N-substitution with adrenaline as follows:

3. In order to prevent, cure, or alleviate the abnormal neurobehavioural conditions and diseases such as hyperactivity, depression, amphetamine, cocaine, ketamine, phencyclidine, and opioid dependance, alcohol abuse, and alcoholism, states of hyper-S-adenosyl-homocystenemia such as chronic renal failure or megaloblastic anemia and Alzheimer's disease that would otherwise ensue from the said defect or abnormality in monoamines already detectable and pharmacologically and therapeutically approachable during the two last intrauterine and the first four postnatal months quinolines are N-substituted with adrenaline. In the said characteristic compositions of matter the active essential ingredients or agents are the methylamine-moiety that is common to the molecule structures of the physiologic hormone adrenaline and the quinoline-ring, characteristically, N-derivated with adrenaline as depicted by the structural formula in (1.1), and the pharmaceutically acceptable salts, prodrugs, and metabolites as well as chiral drugs of this new molecule. Since the compositions of the radicals Ri, Rj, Rk, Rl, Rm, Rn and Ro attached to the structural formula (1.1) are not important for the activity of the above said novel molecule except the adrenaline-moiety, their composition can vary. They can be alternatively H+, alkyl-moieties or ethyl-hydroxyl moieties preferrably shorter or of the same length as 3 carbons including propyl, and isopropyl carbon chains or methoxy-moieties with different interrelating positions with each of the radicals Ri, Rj, Rk, Rl, Rm, Rn and Ro. If alkyl moieties are methyl-groups the carbons of the methyl-moieties can be either mono-, di- or trisubstituted with chloride, iodide or bromide. Instead of hydrogen can exist a chloride, fluoride, iodide or bromide in the quinoline. Further possibilities for the alternatives of hydrogen in the radicals can exist but are not important for the intended use. The moieties can exist in different combinations. The quinoline-ring is substituted with the methyl-amine moiety of adrenaline to develop the characteristic adrenaline-N-quinolines acting more like endogenous non-methylated amines (dopamine, noradrenaline, serotonin, histamine) than like methylated amines (adrenaline) and inhibiting the effect of a pathogenetically and pathophysiologically caused excessive amount of N-methylated amines such as the endogenous adrenaline with known adverse effects on health, returning by their intrinsic sympathomimetic activity and monoamine-mimetic effects the sleep-waking cycles physiologic by inducing more physiologic-like regulations of sleep-waking cycles and especially by affecting the proportion of time human fetuses and babies spend in active sleep, resulting in the prevention, counteracting, and alleviation of the endogenous, intraindividual physiologic and psychologic, interindividual and physio-psycho-social-universal signs and symptoms through improved mental contents (thinking) and emotional states, as a result of pure agonism of beta-1 adrenoceptors, and in the same molecule, pure antagonism of alpha-1 receptors; together with a moderate additional but not necessary property of alpha-2 adrenoceptor blockade resulting in a combination of the property of alpha-1 and alpha-2 adrenoceptors blockade causing an increased ratio of postsypaptic beta-1/alpha-1 adrenoceptors activations ratio at various rates of noradrenergic and adrenergic neuronal functioning, and preferably in the same molecule possibly but not necessarily some of the property of phenyl-ethanol-amine-N-methyl-transferase (PNMT) inhibition, in order to counteract the excessive effect of adrenaline released from the adrenal medullas, for manufacturing, offering for sale, selling, marketing, using, and/or importing, to treat optimally during the last two intrauterine months and the first four postnatal months, and possibly later, though less optimally, the signs and symptoms of later-life neurobehavioural disorders such as hyperactivity occurring predominantly during childhood in attention deficit disorder, amphetamine, cocaine, ketamine, phencyclidine and opioid dependance, depression occurring predominantly in adult women, and alcohol abuse and alcoholism occurring predominantly in adult men, and in states of hyper-S-adenosyl-homocystenemia and Alzheimer's disease. The quinoline-ring structure avoids the harmful effects common in the class of methyl-amines which otherwise would preclude the clinical pharmacologic and therapeutic use of chemical compounds of this novel class of medicines, quinoline-N-adrenalines, and their pharmaceutically acceptable formulations, prodrugs, metabolites, and chiral drugs. In the catecholamine-moiety the preservation of the catechol-groups is important for the intrinsic sympathomimetic activity for the intended therapeutic effects.

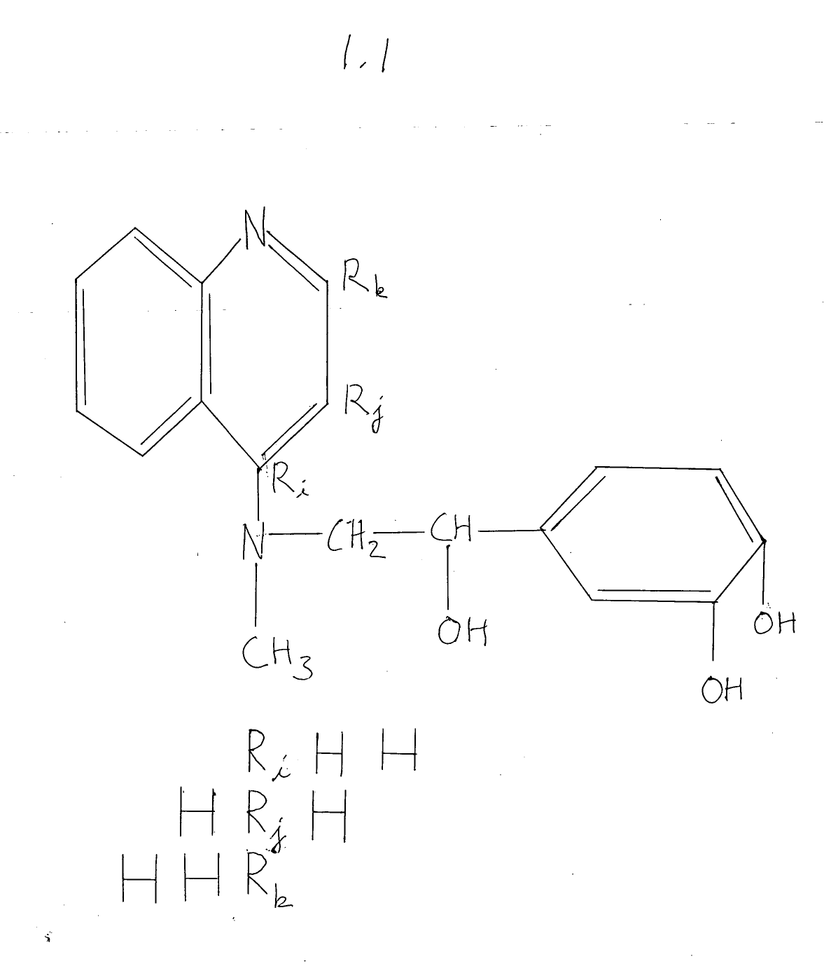

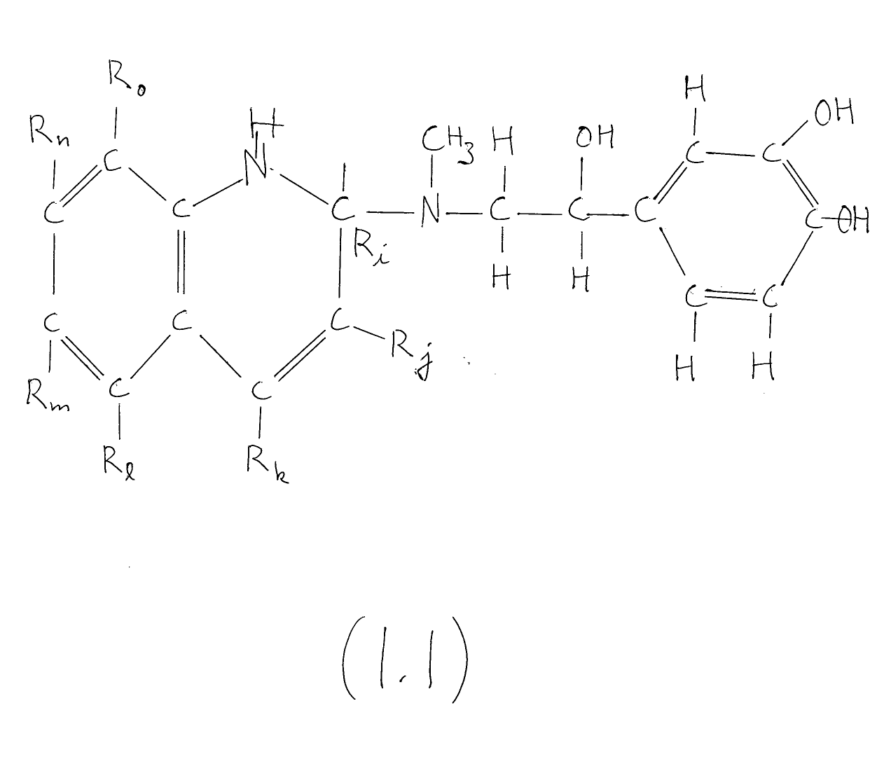

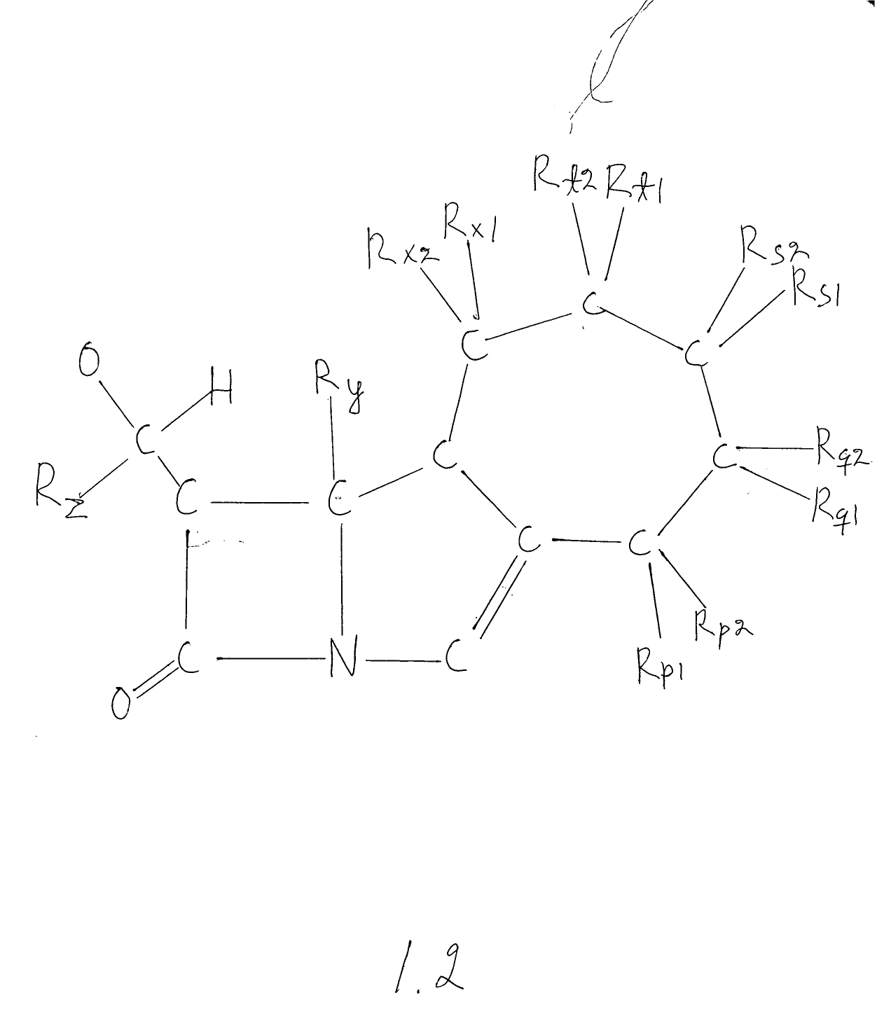

4. The manufacturing, offering for sale, selling, marketing, importing or using of trinems as in the FIG. 1.2 in which the third ring of the tricyclic ring system can be either 5, 6 or 7 carbon ring with substitutions as indicated with Rp 1 and 2, Rq 1 and 2, Rs 1 and 2, Rt 1 and 2, Rx 1 and 2 and Ry, or pharmaceutically acceptable formulations, prodrugs, metabolites, and chiral drugs thereof, as in the claim 3, for the therapeutic purposes as in the claim 3. The substitutions indicated in the FIG. 1.2 as Rp1,2, Rq1,2, Rs1,2, Rt1,2, Rx1,2 and Ry in the cyclic 7-carbon ring can be as said for quinolines in the claim 3 one of them being the characteristic N-adrenaline. Moiety Ry can be either H+ or —N-adrenaline. Moiety Rz can be an alkyl-group. In the cyclic 5-, 6- or 7-carbon ring N-adrenaline can be either in the position 1 or 2 in Rp1,2, Rq1,2, Rs1,2, Rt1,2 and Rx1,2. The moieties in the positions 1 and 2 can be the same or different from each other, and they can exist in different combinations in relation with the moieties next to them. Instead of positions 1 and 2 can exist acetyl- or oxygen-moiety.

5. The manufacturing, offering, selling, marketing, importing, or using, of chemical compounds, and pharmaceutically acceptable formulations, prodrugs, metabolites, and chiral drugs that inhibit phenyl-ethanol-amine transferase (PNMT) not necessarily passing through the blood-central nervous system-barrier or if so acting on adrenergic neurotransmissions in the brain stems of human fetuses and newborns, with a possible but not necessary, moderate limitation of S-adenosylmethionine in the diet, during the last two intrauterine months, and of human babies during the first four postnatal months, to induce a reduction in the synthesis and storage of adrenaline that is available for release into the blood stream from the adrenal medullas with effects dominantly on beta-1 and alpha-2 adrenergic receptors, on alpha-1 receptors that are dominantly noradrenergic receptors, and on beta-2 receptors, and other adrenergic receptors such as beta-3 receptors, for a use solely for the indications as in previous claims.

6. The manufacturing, offering for sale, selling, marketing, importing or using of chemical compounds, and pharmaceutically acceptable formulations, prodrugs, metabolites, and chiral drugs as in the claims 3 and 4 to be given during the last two intrauterine and the first four postnatal months, and less optimally later, for improving impairments in the states of hyper-S-adenosylhomocystenemia such as chronic renal failure or megaloblastic anemia and Alzheimer's disease during later life.

7. Allowing for pharmacokinetic and pharmaceutical interactions concerning different pharmaceutically acceptable formulations and routes of administration the two separate molecules—a characteristic PNMT-inhibitor and a characteristic N-adrenaline—can be combined. The drug developments are used to treat neurobehavioural symptoms of later life conditions and diseases such as childhood hyperactivity, amphetamine, cocaine, ketamine, phencyclidine, and opioid dependance, predominantly in women occurring depression, and predominantly in men occurring alcohol abuse and alcoholism, and states of hyper-S-adenosyl-homocystenemia such as chronic renal failure or megaloblastic anemia and Alzheimer's disease.

8. The manufacturing, offering for sale, selling, marketing, importing or using of 5-N-adrenaline-ciprofloxacine (1-cyclopropyl-6-fluoro-7N-adrenaline-1,4-dihydro-4-oxo-7-(1-piperazinyl)-3-quinolinecarboxylic acid) or 5-N-adrenaline-moxifloxacin (1-cyclopropyl-5-N-adrenaline-6-fluoro-1,4-dihydro-8-methoxy-7-((4aS,7aS)-octahydro-6H-pyrrolo(3,4-b)pyridin-6-yl)-4-oxo-3-quinolinecarboxylic acid) or 8-N-adrenaline-ofloxacin (8-N-adrenaline-9-fluoro-2,3-dihydro-3-methyl-10-(4-methyl-1-piperazinyl)-7-oxo-7H-pyrido(1,2,3-de)-1,4-benzoxazine-6-carboxylic acid) given during the last two intrauterine and the first four extrauterine months of human ontogeny, and less optimally later during ontogeny, to treat later life hyperactivity (of attention deficit disorder), depression, drug dependence and drug abuse including amphetamine, cocaine, ketamine, phencyclidine and opioid dependence and abuse, alcohol abuse and alcoholism and states of hyper-S-adenosylhomocystenemia such as chronic renal failure and megaloblastic anemia.

9. Pertinent to claim 3, the manufacturing, offering for sale, selling, marketing, importing or using of 4-hydro-isoquinoline-N-adrenalines given during the last two intrauterine and the first four extrauterine months of human ontogeny, and less optimally later during ontogeny, to treat later life hyperactivity (of attention deficit disorder), depression, drug dependence and drug abuse including amphetamine, cocaine, ketamine, phencyclidine and opioid dependence and abuse, alcohol abuse and alcoholism and states of hyper-S-adenosylhomocystenemia such as chronic renal failure and megaloblastic anemia.

10. The manufacturing, offering for sale, selling, marketing, importing or using 2- or 3- or 4- or 5- or 6-benzyl-penicillium-N-adrenaline or, respectively, (2S,5R,6R)-3,3-dimethyl-7-oxo-6-(2- or 3- or 4- or 5- or 6-phenylacetyl)amino)-4-thia-1-azabicyclo(3.2.0)heptane-2-carboxylic acid compound with N-(phenylmethyl)benzeneethaneamine (1:1), or (2S,5R,6R)-3,3-dimethyl-7-oxo-6-(((2- or 3- or 4- or 5- or 6-N-adrenaline)phenylacetyl)amino)-4-thia-1-azabicyclo(3.2.0)heptane-2-carboxylic acid compound with N,N′-bis(phenylmethyl)-1,2-ethanediamine (2:1) which is N-adrenaline of penicillin G benzathine, or (2S,5R,6R)-3,3-dimethyl-7-oxo-6-(((2- or 3- or 4- or 5- or 6-N-adrenaline)phenylacetyl)amino)-4-thia-1-azabicyclo(3.2.0)heptane-2-carboxylic acid compound with 2-(diethylamino)ethyl 4-aminobenzoate (1:1) monohydrate which is penicillin G procain-N-adrenaline, or (2S,5R,6R)-3,3-dimethyl-7-oxo-6-(((2-propenyl-thio(propenyl(1-N-adrenaline)))acetyl)amino)-4-thia-1-azabicyclo-(3.2.0)heptane-2-carboxylic acid or, respectively, propenyl(2-N-adrenaline) or propenyl(3-N-adrenaline), which is propenyl-penicillin O-1-, 2- or 3-N-adrenaline, or 2- or 3- or 4- or 5- or 6-(2S,5R,6R)-3,3-dimethyl-7-oxo-6-((fenoxyacetyl)amino)-4-thia-1-azabicyclo(3.2.0)heptane-2-carboxylic acid —N-adrenaline, which is 2- or 3- or 4- or 5- or 6-(phenoxyacetyl)-(penicillium V)-N-adrenaline, given during the last two intrauterine and the first four extrauterine months of human ontogeny and less optimally later for treatment of the later life diagnoses as in the claims 1,2 and 3.

11. The manufacturing, offering for sale, selling, marketing, importing or using 2- or 3- or 4- or 5- or 6-N-adrenaline-(6R,7R)-7-(((2R)-Aminophenylacetyl)amino)-3-methyl-8-oxo-5-thia-1-azabicyclo(4.2.0)oct-2-ene-2 carboxylic acid, which is cephalexin-N-adrenaline, given during the last two intrauterine and the first four extrauterine months of human ontogeny and less optimally later for treatment of the later life diagnoses as in the claims 1, 2 and 3.
